(19) United States
(12) Patent Application Publication
(10) Pub. No.: US 2019/0209748 A1
(43) Pub. Date: Jul. 11, 2019

(54) BREAST PUMP VACUUM PUMP AND CARRYING CASE

(71) Applicant: Moxxly, Inc., San Francisco, CA (US)

(72) Inventors: Santhi Analytis, San Francisco, CA (US); Cara Delzer, San Francisco, CA (US); Gabrielle Guthrie, San Francisco, CA (US); Wisit Jirattigalachote, San Francisco, CA (US); Jacob Kurzrock, San Francisco, CA (US); Claire O'Malley, San Francisco, CA (US)

(73) Assignee: Moxxly, Inc., San Francisco, CA (US)

(21) Appl. No.: 16/244,034

(22) Filed: Jan. 9, 2019

Related U.S. Application Data
(60) Provisional application No. 62/615,221, filed on Jan. 9, 2018.

(51) Int. Cl. A61M 1/06

(52) U.S. Cl. CPC A61M 1/062; A61M 2205/3334; A61M 2205/3576; A61M 2205/3379; A61M 2209/06; A61M 2205/502; A61M 2205/8206; A61M 2205/8237; A61M 2205/3673; A61M 2205/3365

(57) ABSTRACT

A breast pump suction pump is provided. The breast pump suction pump may include one or more sensors. The breast pump suction pump may further include a processor. The one or more sensors may provide sensor data to the processor and, in response, the processor may automatically adjust a pump parameter of the breast pump suction pump.

### CROSS-REFERENCE TO RELATED APPLICATIONS

This application claims the benefit of U.S. Provisional Patent Application Ser. No. 62/615,221, filed Jan. 9, 2018, entitled “BREAST PUMP VACUUM PUMP AND CARRYING CASE,” which is incorporated herein by reference in its entirety, including but not limited to those portions that specifically appear hereinafter, the incorporation by reference being made with the following exception: In the event that any portion of the above-referenced application is inconsistent with this application, this application supercedes said portion of said above-referenced application.

### BACKGROUND

### 1. Technical Field

This disclosure relates generally to a breast pump for nursing mothers. More specifically, the devices and systems disclosed herein relate to a vacuum pump that provides vacuum suction to the breast pump and a carrying case, or clutch, that contains parts from a breast pump when not in use and that warms and cools milk.

### 2. Description of the Related Art

Nursing an infant can be an emotional experience for many nursing mothers. While nursing can be profoundly rewarding for a new mother, many mothers have difficulty nursing a new baby. Breast pumps have been developed to help women express milk by mechanical systems to feed to their babies. To that end, breast pumps have been developed to not only assist mothers who experience difficulty in nursing infants, but also to allow mothers who are away from their babies provide milk for their infants to drink at another time and help many mothers maintain and develop their milk supply.

Conventional breast pump systems are not well suited for today's user. Many of these breast pumps include an oppressively large pump system that connects to the breast pump by pneumatic tubing. These large pumps are typically carried in a carrying case with a shoulder strap so a nursing mother can transport the pump. Many times, the pumps are not intended to be removed from the carrying case during use. Thus, flaps are installed in the carrying case to provide the user with access to pump controls, battery replacement trays, and charging ports.

Conventional pumps are merely pumps that generate suction. They provide no information on the pumping session. These pumps are not interactive nor do they provide feedback on the effectiveness of the pumping session.

Another further problem is that once milk has been pumped, the milk must be stored in a way that prevents it from spoiling. If the milk is not to be immediately consumed after pumping, it is generally accepted that the milk should be cooled for food safety. At the same time, however, nursing infants typically will not drink cold milk. Conventionally, mothers have relied on different kitchen appliances or hot water baths to warm milk that has been cooled or frozen. But, this requires that the mother have access to kitchen appliances or some hot water source, which may not always be possible.

It is therefore one object of this disclosure to provide a breast pump that provides real-time information on a pumping session. It is a further object of this disclosure to provide a clutch that maintains all of the parts associated with a breast pump. Another object of this disclosure is to provide a clutch that maintains all of the parts associated with a breast pump and implements a Peltier tile to heat and cool milk stored within the clutch.

### SUMMARY

Disclosed herein is a breast pump suction pump. The breast pump suction pump may include one or more sensors. The breast pump suction pump may further include a processor. The one or more sensors may provide sensor data to the processor and, in response, the processor may automatically adjust a pump parameter of the breast pump suction pump.

Also disclosed herein is a breast pump suction pump system. The breast pump suction pump system includes a breast pump suction pump. The breast pump suction pump includes one or more sensors and a processor receiving sensor data from the one or more sensors. The breast pump suction pump system further includes a mobile device. The processor may transmit sensor data to the mobile device and receive control instructions from the mobile device.

### BRIEF DESCRIPTION OF THE DRAWINGS

The accompanying drawings illustrate one or more embodiments of a breast pump suction pump and a clutch for maintaining the parts associated with the breast pump.

FIG. 1 illustrates a breast pump suction pump.

FIG. 2 illustrates a breast pump suction pump system.

FIG. 3 illustrates an embodiment of the clutch.

FIG. 4 illustrates an internal view of the clutch.

FIG. 5 illustrates a Peltier tile element.

FIG. 6 illustrates a Peltier tile element disposed within the clutch.

### DETAILED DESCRIPTION OF PREFERRED EMBODIMENTS

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

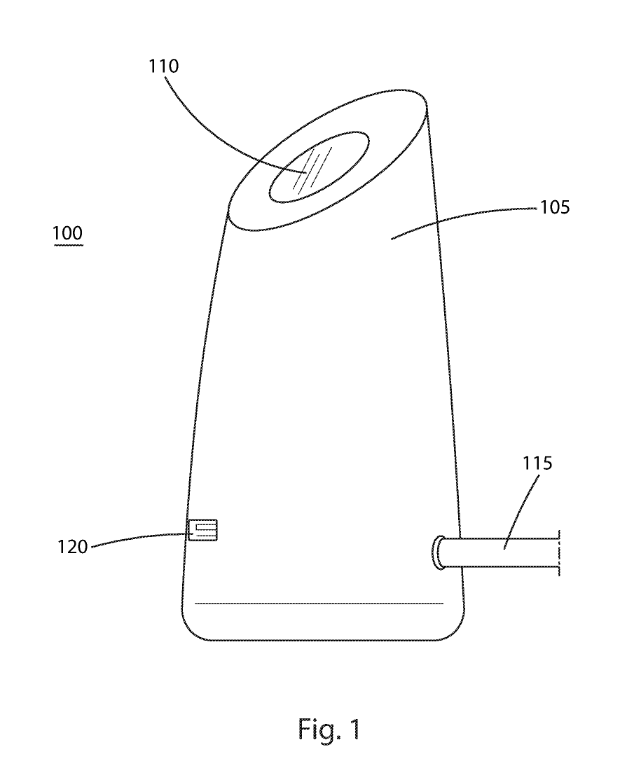

FIG. 1 illustrates a breast pump suction pump 100. Suction pump 100 includes a housing 105 which includes a display screen 110. Housing 105 is typically constructed using hard plastic while display screen 110 can be implemented as an LCD, LED, OLED, ELD, PDP or any other display screen. The screen may be touch-operated, such as a resistive or capacitive touch screen. Display screen 110 may further be implemented to display a series of graphical elements as colored lights representative of flow rates. For example, a red light in display screen 110 may indicate an unsatisfactory milk pumping flow rate while a yellow light in display screen 110 indicates a milk pumping flow rate that meets minimum thresholds. A green light in display screen 110, for example, may indicate that a mother is pumping at a sufficient milk pumping flow rate to produce a desired amount of milk. Other colors of pulsing and/or fading lights and icons may be implemented in a way that provides pumping feedback to a mother. Further, display screen 110 may display a flow rate in a unit per time format (e.g., ounces per hour). Display screen 110 may further include a clock or any other information relevant to a pumping session. Suction pump 100 may further react to feedback received from sensors, such as those that will be described below. In one embodiment, the suction pump may adjust suction power or other parameter of operation to enhance the effectiveness of a particular pumping session.

Suction pump 100 includes a pneumatic tubing 115 that a user may attach to a breast pump. Suction pump 100 may further include a retraction system to retract pneumatic tubing 115 into suction pump 100 at the end of the pumping system. For example, pneumatic tubing 115 may be loaded on a spring loaded reel inside suction pump 100. As pneumatic tubing 115 is drawn out of suction pump 100, the spring loaded reel catches on specific points within the suction pump 100 to allow the pneumatic tubing to be held out of suction pump 100. Once a pumping session has completed, the user may be able to slightly pull on the pneumatic tubing to disengage the catch points and allow the spring loaded reel to wind the pneumatic tubing back onto the spool in suction pump 100.

Finally, breast pump 100 includes a charging port 120. In FIG. 1, charging port 120 is shown as a USB charging port. However, any type of electrical connection is suitable. Examples may include a barrel connector, mini USB, micro USB, USB-A, USB-B, or any other connector suitable for carrying electrical power to suction pump 100. Further, suction pump 100 may include a battery that may be charged through charging port 120. Thus, breast pump 100 may be used on battery power or direct electrical power through charging port 120, allowing breast pump 100 to be used nearly anywhere.

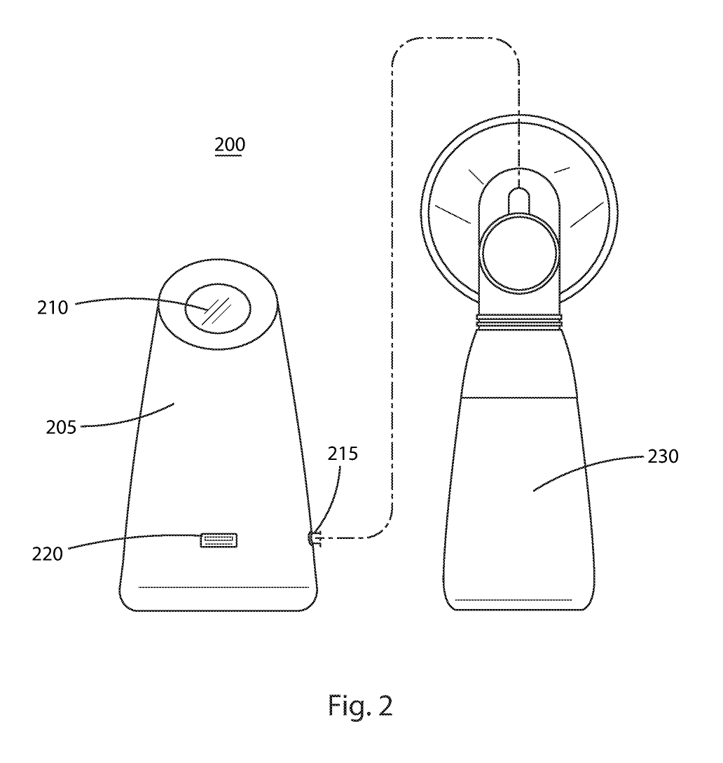

FIG. 2 illustrates a breast pump suction pump system 200. Breast pump suction pump system 200 includes a suction pump 200, which is similar in description and implementation to suction pump 100, shown in FIG. 1. Suction pump 200 further includes a housing 205, display screen 210, pneumatic tubing 215, and charging port 220, each of which are similar in implementation and description to the corresponding element shown in FIG. 1. Breast pump suction pump system 200 further includes a breast pump 230.

In the embodiment of FIG. 2, pneumatic tubing 215 is connected to breast pump 230. Pneumatic tubing 215 allows suction created by suction pump 200 to create a vacuum within a manifold of breast pump 230 to simulate the suction pressure of a suckling infant. The body-worn breast pump 230 may be air sealed to a mother's breast such that a vacuum may form within the manifold and assist the mother in expressing milk. Milk that is expressed may be caught by breast pump 230 without flowing into or blocking pneumatic tubing 215.

One or more sensors and processors associated therewith may be implemented within breast pump 230 that may transmit information to a user's mobile device and/or suction pump 200. For example, breast pump 230 may include a flow rate sensor which communicates flow rate information to suction pump 200 for display on display screen 210. Flow rate information may include the number of milk droplets detected, a true flow rate, or any other information necessary to provide a flow rate or a total volume of milk that has been detected by the sensors implemented in breast pump 230. Other sensors may be implemented within suction pump 200. For example, pressure sensors may be implemented inside suction pump 200 which detect an amount of suction pressure (pounds per square inch, for example) applied to a breast. Sensor information may be provided to the user by a touch screen or tactile display 210.

The pump motor 200 may be controlled by the user directly by touch-screen or tactile display 210, else the pump motor 200 may be controlled through the user's mobile device by the user. Alternatively, pre-set controls for the pump operation can be sent from the mobile device to the pump. Alternatively, the pump settings or pump parameters, may be changed based on sensor data as sent from the mobile device, whether provided manually by a user or automatically by a mobile device. For example, if the user's flow rate is low, the pump can automatically increase suction, pump cycle speed or other pump parameters. Depending on the performance of each breast, as measured by the sensors, the pump can be individually controlled so each side has a different pumping profile. Historical trends in volume and flow rate of milk produced (e.g., a user pumping profile) may be displayed on the touch screen or tactile display 210. Similarly, corresponding suction pressure applied by pump motor 200 in real time and historical trends relative to volume and flow rate of milk produced, may be provided by display on touch screen or tactile display 210.

Pump motor 200 may have a battery charger charging the battery when it is removed from pump motor 200, or may charge the battery by port 220. The battery is removable and can be charged separately from the motor. Also, the motor may be used during charging, while a power cable is attached to the charging port 220.

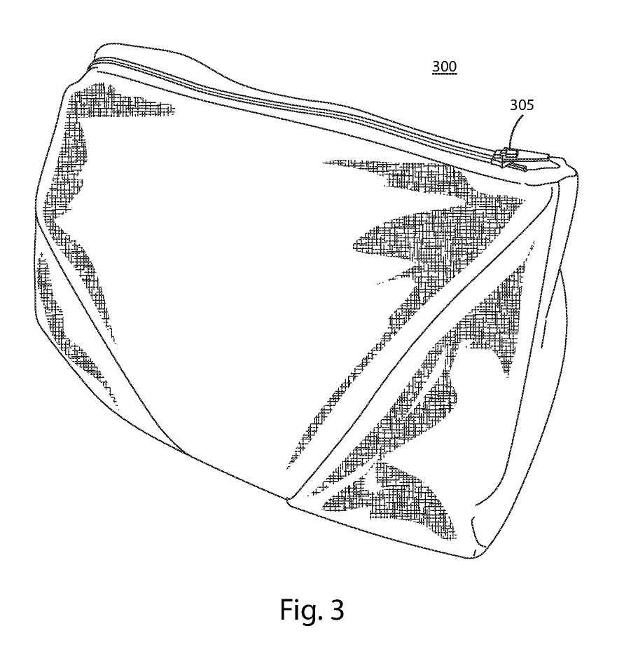

FIG. 3 illustrates one embodiment of a clutch 300. Clutch 300 is essentially a carrying case for breast pump suction pump system 200, shown in FIG. 2. Clutch 300 includes an opening 305 for accessing an internal portion of clutch 300. Opening 305 is shown as being operated by a zipper but could be implemented using any known connector. The clutch may incorporate a charging system, back up battery pack, and/or charging cable to charge and operate the motor and/or sensors.

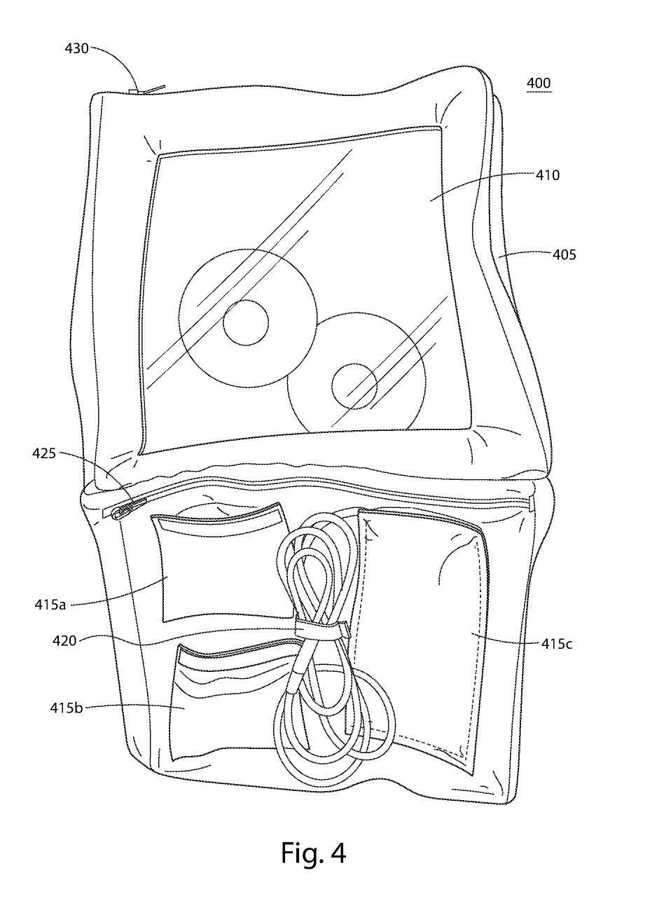

FIG. 4 illustrates an internal view of clutch 400 which is similar in implementation and description to clutch 300 shown in FIG. 3. Clutch 400 includes an external portion 405 and an internal portion 410. External portion 405 may include a loop for hanging clutch 400 on a hanger. Alternatively, a loop or hook may be located at the top edge of the inner portion. Alternatively a long handle may expand the internal and external portions to change the method of carrying the case, such as between a hand-bag and cross-body bag. Internal portion 410 provides for a number of pockets such as pockets 415a, 415b, and 415c which are each configured to hold various parts of breast pump suction pump system 200, shown in FIG. 2. Internal portion 410 further includes a pocket with a clear plastic cover for larger parts, such one or more breast pump flanges, bottles, etc. Clutch 400 may further include a retaining strap 420 for retaining coiled loops of pneumatic tubing. Pocket 425 may include a heating and cooling element for warming milk and cooling milk, as will be described below. Pocket 425 may further include a rechargeable battery for charging suction pump 200 shown in FIG. 2 or operating a Peltier tile, as will be discussed below. Opening 430 is similar to opening 305 in FIG. 3 and allows the edges of clutch 400 to be connected together, as shown in FIG. 3.

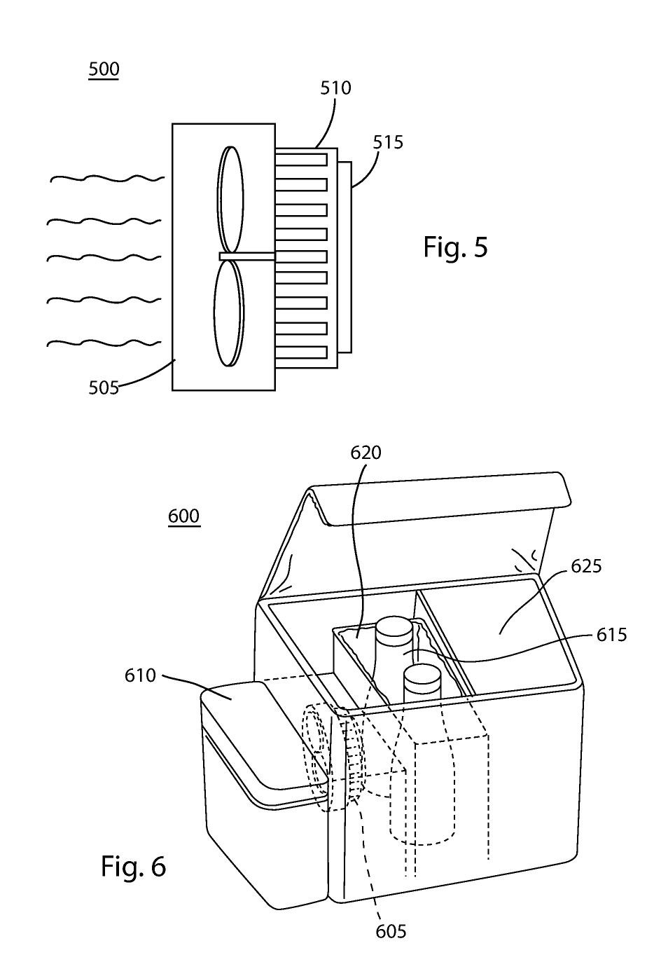

FIG. 5 illustrates a Peltier tile element 500 for use within clutch 300, shown in FIG. 3 or clutch 400 shown in FIG. 4, for example. Peltier tile element 500 includes an exhaust fan 505 to pull heat through heat sink 510 and Peltier tile 515. Peltier tile 515 allows an electrical current to flow between a junction of two conductors. Heat is removed from the junction by heat sink 510 and exhausted by exhaust fan 505. Peltier tile 515 may include thousands of such junctions that allow heat to be removed from an enclosed space which results in a cooling effect in that space. In addition, or alternatively, Peltier tile element 500 may include another heating element such as copper wire or ceramic resistors. In other words, one side of Peltier tile 515 may be cooled to between 1° and 4° Celsius, depending on the ambient temperature outside the bag. The other side of Peltier tile 515 may be heated by the exhausted heat up to approximately body temperature.

FIG. 6 illustrates a Peltier tile element 605 disposed within clutch 600. Clutch 600 may be similar in implementation and description to Clutch 300 shown in FIG. 3 and clutch 400 shown in FIG. 4. However, in this case, clutch 600 includes a heating pouch 610, an insulated pouch 615, an insulating liner 620, and a storage pouch 625 for storing parts and accessories associated with a breast pump.

Peltier tile element 605, which is similar in implementation and description to Peltier tile element 500, discussed above, may be disposed between insulating pouch 620 and heating pouch 610. Thus, when an electrical current is applied to Peltier tile element 605, as described above, a cooling effect occurs on a cooling side of Peltier element 605. The cooling effect is targeted on insulating pouch 620 while the heating effect, the heat pulled through the heat sink by the fan in Peltier tile element 605, is targeted into heating pouch 610. Thus, a mother may store milk in a bottle or bag within insulating pouch 620 in an effort to cool the milk for later use. Later, a mother may put previously pumped milk into heating pouch 610 in order to warm the milk for her baby's consumption.

In one embodiment, heating pouch 610, insulated pouch 615, and storage pouch 625 may include adjustable compartments, using elastic rims, for example. This allows for the heating for milk in bottles or freezer bags, or milk receptacles of varying sizes. Heating pouch 610 and insulated pouch 615 may be further implemented within clutch 600 using temperature controlling fabrics to regulate the temperature of a pouch. One example of temperature controlling fabrics include a water lining or a wax lining, or another embedded textile. Storage pouch 625 of clutch 600 may be further divided to include compartments that correspond to breast pump accessories, a breast pump suction pump, and a power supply with an electrical plug or portable battery. Finally, because, for example, the breast pump suction pump, the power supply, and other electrical based elements can be sensitive to water, storage pouch 625 may include water resistant compartments to ensure electrical based elements stay dry within clutch 600.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A breast pump suction pump, comprising:
one or more sensors, and
a processor, wherein the one or more sensors provide sensor data to the processor and, in response, the processor automatically adjusts a pump parameter of the breast pump suction pump.

2. The breast pump suction pump of claim 1, wherein the pump parameter is a suction power applied by the breast pump suction pump.

3. The breast pump suction pump of claim 1, wherein the pump parameter is a pump cycle speed.

4. The breast pump suction pump of claim 1, wherein the sensor data includes information about suction power applied by the breast pump suction pump.

5. The breast pump suction pump of claim 1, wherein the processor may further receive data from one or more of a breast pump and a mobile device.

6. The breast pump suction pump of claim 5, wherein the data received from one or more of the breast pump and the mobile device includes milk flow rate information.

7. The breast pump suction pump of claim 5, wherein the data received from one or more of the breast pump and the mobile device includes pumped milk volume information.

8. The breast pump suction pump of claim 1, further comprising, a display.

9. The breast pump suction pump of claim 8, wherein the display provides real-time pumping information.

10. The breast pump suction pump of claim 9, wherein the real-time pumping information includes one or more of suction pump pressure, volume of milk pumped, and milk flow rate.

11. The breast pump suction pump of claim 8, wherein a graphical element on the display indicates a milk flow rate.

12. The breast pump suction pump of claim 8, wherein the display provides real-time pumping information on a per-breast basis or on a total volume basis.

13. The breast pump suction pump of claim 1, wherein automatically adjusting a pump parameter is based on historical pumping information.

14. The breast pump suction pump of claim 1, wherein the breast pump suction pump is wirelessly connectable to a mobile device to receive control instructions from the mobile device.

15. The breast pump suction pump of claim 1, wherein the breast pump suction pump includes a removable battery.

16. The breast pump suction pump of claim 15, further comprising a battery charger that charges the removable battery.

17. A breast pump suction pump system, comprising:
a breast pump suction pump, comprising:
one or more sensors, and
a processor receiving sensor data from the one or more sensors; and
a mobile device;
wherein the processor may transmit the sensor data to the mobile device and receive control instructions from the mobile device.

18. The breast pump suction pump system of claim 17, wherein the control instructions include breast pump suction pump parameter adjustments.

19. The breast pump suction pump system of claim 18, wherein the suction pump parameter adjustments are received by the processor and are based on one of manual input from the user and or automatic adjustments based on a user pumping profile.

20. The breast pump suction pump system of claim 18, wherein the suction pump parameter adjustments are received by the processor and based on automatic instructions from the mobile device.
